(19) United States
(12) Patent Application Publication
(10) Pub. No.: US 2004/0018526 A1
(43) Pub. Date: Jan. 29, 2004

(54) Method of detecting abasic sites on DNA

(75) Inventors: Tamaki Hirose, Gunma (JP); Atsushi Tanaka, Gunma (JP)

Correspondence Address: BANNER & WITCOFF, 1001 G STREET N W, SUITE 1100, WASHINGTON, DC, 20001, US

(73) Assignee: Japan Atomic Energy Research Institute, Kashiwa-shi (JP)

(21) Appl. No.: 10/437,283

(22) Filed: May 14, 2003

(30) Foreign Application Priority Data
Jul. 24, 2002 (JP) 2002-215112

(51) Int. Cl. C12Q 1/68; C12M 1/34

(52) U.S. Cl. 435/6; 435/287.2

(57) ABSTRACT

The present invention relates to a method for visual detection of abasic sites on DNA, comprising the steps of (i) labeling abasic sites on DNA, (ii) fixing a linear form of the labelled DNA onto a substrate, and (iii) subsequently detecting the respective abasic sites on the fixed labelled DNA. The method can be implemented by an apparatus for visual detection of abasic sites on DNA, which comprises (i) a fixing means by which a labelled DNA sample having labelled abasic sites on DNA is fixed in linear form onto a substrate and (ii) a detecting means for detecting the respective labelled abasic sites.

### BACKGROUND OF THE INVENTION

This invention relates to a method in which abasic sites on DNA that occur as the result of DNA damage or mutation are labelled and detected both qualitatively and quantitatively.

DNA damage which occurs as the result of exposure to certain chemical species, irradiation with X-rays and ultraviolet rays or exposure to oxidizing stress can be a major cause of mutation and carcinogenesis. Abasic sites constitute a common type of damage that occurs in DNA molecules and they result from the dropping of bases on one of the two strands in double-stranded DNA. It is known that abasic sites are formed in both plants and animals by spontaneous depurination, ionizing radiation or repair (Atamna, H. et al. (2000) Proc. Natl. Acad. Sci., 97, 685-691; Dandoy, E. et al. (1987) Mutat. Res., 181, 57-60; Friedberg, E.C. et al. (1995) DNA Repair and Mutagenesis, ASM Press, Washington D.C.).

Abasic sites constitute one of the most predominant kinds of DNA damage and detecting them is essential to diagnosis of cancer and immunodeficiency that result from DNA damage. A number of techniques have been developed to detect abasic sites and they include (1) the methoxyamine method which uses ¹⁴C-methoxyamine to label abasic sites radioactively (such as radio isotope), (2) the enzyme assay which is performed by binding an enzyme conjugate to an aldehyde-reactive probe (ARP) which binds specifically to abasic sites, (3) the liposome method using fluorochrome-containing liposomes as ARP, and (4) atomic force microscopy which uses an atomic force microscope to provide an image of ARP-bound avidin.

The methoxyamine method takes advantage of the fact that methoxyamine and an abasic site react at a substantially quantitative ratio of 1:1 to form a stable linkage. Thus, ¹⁴C-methoxyamine is bound to the abasic site and later detected (Talpaert-Borle, M & Liuzzi, M. (1983) Biochem. Biophys. Acta 740, 410-416). However, this method has two problems, one being the extremely low specific activity of ¹⁴C-methoxyamine which leads to low sensitivity for the quantification of abasic sites and the other being the inability to visualize the abasic site since it is labelled with ¹⁴C.

In the enzyme assay, a biotin-containing aldehyde-reactive probe (ARP) is specifically bound to abasic damaged sites in DNA and the resulting specific linkage between biotin and an avidin-enzyme conjugate is employed to induce color development of the color forming substrate in solution by reaction with the enzyme in the avidin-enzyme conjugate (Kubo, K. et al. (1992) Biochemistry, 31, 3703-3708). This method, which is extensively used today, has high sensitivity and achieves reliable quantification. However, since the method measures the average quantity of abasic sites on DNA, individual DNA molecules cannot be specified in terms of the number and location of abasic sites they have and it is impossible to detect such abasic sites visually.

In the liposome method, ARP is bound to the surfaces of liposomes containing a fluorochrome and abasic sites can be detected with a very small quantity of DNA, about a thousandth of the quantity required in the enzyme assay (see the article posted by Sugawara, M. et al. at the 50th Annual Meeting of the Japan Society for Analytical Chemistry, http://wwwsoc.nii.ac.jp/jsac/TT50/3F21.html). However, the liposome method also involves quantification of abasic sites in total DNA, so individual DNA molecules cannot be specified in terms of the number and location of abasic sites they have and it is impossible to detect such abasic sites visually.

In the atomic force microscopy, the interatomic force (either attractive or repulsive) between a surface of an object and a tiny probe tip scanning it is detected and subjected to image processing with a computer (Sun, H. B. et al. (2001) Anal. Chem., 73, 2229-2232). However, this method needs an expensive apparatus and is only applicable to specialized situations. Another problem with the method is that it permits observation of only dried DNA samples. As a further disadvantage, the atomic force microscopy enables detection of only very narrow regions of DNA with lengths from several to several hundred bases, so it is impossible to quantify and identify abasic sites present on large DNA molecules such as chromosomes.

Thus, there has been no method in the prior art that tells how many abasic sites occur and in which areas of DNA as it is taken either partially or as a whole and abasic sites that can lead to mutation, carcinogenesis or aging cannot be diagnosed at the molecular level.

### SUMMARY OF THE INVENTION

An object of the present invention is to provide a method by which abasic sites occurring on DNA are individually detected in a visible manner so that their number and location on the DNA can be determined.

As the result of intensive studies they conducted to solve the aforementioned problems of the prior art, they found that the stated object can be accomplished by adopting the following design.

In its first aspect, the present invention provides a method for visual detection of abasic sites on DNA, which comprises the steps of:

(i) labeling abasic sites on DNA; and

(ii) fixing a linear form of the labelled DNA onto a substrate; and

(iii) subsequently detecting the respective abasic sites on the fixed labelled DNA.

In this method, the order of steps (i) and (ii) is not fixed and either may precede the other.

In the present invention, visual detection of the abasic sites on DNA can be realized by labeling them with a fluorochrome.

In order to label the abasic sites, its aldehyde group may be labelled. The aldehyde group can be recognized using a hydroxylamine derivative such as methoxyamine or ARP.

In its second aspect, the present invention provides an apparatus for visual detection of abasic sites on DNA, which comprises:

(i) a fixing means by which a labelled DNA sample having labelled abasic sites on DNA is fixed in linear form onto a substrate; and

(ii) a detecting means for detecting the respective labelled abasic sites.

### BRIEF DESCRIPTION OF THE DRAWINGS

FIG. 1 is a graph showing the fluorescence intensity (in arbitrary unit) vs. exposure time of the fluorochrome Cy5 bound streptavidin used in the invention;

FIG. 2 is a histogram in which the fluorescence intensity (in arbitrary unit) of the fluorochrome Cy5 bound streptavidin used in the invention is compared with the fluorescence intensity (in arbitrary unit) of the standard fluorochrome Cy5 bound dUTP;

FIG. 3 is a set of photographs showing the results of visual observation of abasic sites using the standard ARP-DNA having a known number of abasic sites;

FIG. 4 shows a one-field image obtained by direct visual detection of abasic sites on individual DNA molecules;

FIG. 5 is a picture that characterizes the abasic sites on a single DNA molecule as detected by direct visualization according to the invention; and

FIG. 6 is a graph showing that abasic sites on DNA could be quantified almost completely by the invention.

### DETAILED DESCRIPTION OF THE INVENTION

The present invention is described below in detail. The method of the invention for detecting abasic sites on DNA comprises the steps of fluorescently labeling abasic sites that occur as the result of DNA damage and subsequent repair process, linearizing the labelled DNA on a glass plate or other substrate such that the individual DNA molecules are spread and fixed without overlapping, visually observing the fixed DNA molecules by a detection means such as an optical microscope, and measuring their fluorescence intensity to determine the position and number of the abasic sites occurring on the DNA.

The term “abasic sites on DNA” as used herein refers to those sites of DNA from which bases have dropped upon DNA exposure to a certain chemical species, illumination with ultraviolet rays or X-rays, or exposure to oxidizing stresses. The bases that drop from the abasic sites may be purine- or pyrimidine bases, so the concept of abasic sites covers both depurinated and depyrimidinated sites. These abasic sites are produced when DNA is exposed to the above-mentioned external conditions. They can also occur spontaneously under physiological conditions. Another occasion of the production of abasic sites is in the process of repair of other DNA damage such as alkylation of bases. Thus, abasic sites are the most common type of DNA damage. If this type of DNA damage is not repaired appropriately, induced mutation is likely to occur, sometimes leading to carcinogenesis or aging. The present invention provides a method and an apparatus by which individual abasic sites can be recognized as such and detected visually.

Since the present invention aims at visually detecting individual sites of such damage on DNA, the first necessary step is labeling DNA with a detectable marker. The term “visually” usually means detectability with the naked eye under visible light but, in the present invention, detection with an apparatus capable of signal detection with visible light is also intended.

The term “labeling” as used herein means such a process that the step of specifically recognizing abasic sites on DNA and the step of labeling the abasic sites such that they can be detected are performed either simultaneously or consecutively. The recognition and labeling of abasic sites may be performed either directly using a single molecule or indirectly using a combination of two or more molecules.

It is known that abasic sites on DNA can be recognized using reagents that specifically recognize the aldehyde group that occurs when bases have dropped and examples of such reagents are hydroxylamine derivatives (e.g. ARP) and methoxyamine. From the viewpoints of specificity and reactivity, hydroxylamine derivatives are preferably used in the present invention in order to ensure that abasic sites on DNA are recognized specifically.

Abasic sites are labelled with either one of detectable marker molecules. In the present invention, it is preferred to use marker molecules that can be detected visually and fluorochromes are exemplary detectable marker molecules. Any fluorochromes known in the art may be used and examples include fluorescein, rhodamine, Cy3 and Cy5. The most preferred fluorochromes in the invention are Cy5 and Cy3.

The labeling may be either direct or indirect. In direct labeling, the marker molecule is directly coupled to the recognizing portion for abasic sites on DNA. Indirect labeling can be accomplished by binding a plurality of molecules that provide intermolecular specific binding characteristics. This may be accomplished by binding biotin to the abasic site recognizing portion and binding the marker molecule to avidin which is capable of specific binding to biotin, whereby the abasic site recognizing portion is indirectly bound to the marker molecule by means of the avidin-biotin binding characteristics. If desired, molecules other than the avidin-biotin conjugate can also be used to accomplish indirect labeling.

In order to attain the object of individually detecting the respective sites of DNA damage on DNA, the present invention also requires that DNA be fixed in linear form on a substrate.

DNA is said to be “linear” if all sites on a single DNA molecule are in such a state that any of one of them can be detected as an entity that is completely distinguishable from all other sites on the same DNA molecule or from other DNA molecules. In other words, it is required that when DNA is fixed on a substrate, there should be no area of physical overlap either within the same DNA molecule or with other DNA molecules. In a preferred case of the invention, DNA molecules are spaced apart and every single DNA molecule is substantially in linear form.

The substrate onto which DNA is fixed may be made of any material as long as it is clear enough to enable visual detection. Examples include glass, transparent plastics and mica. The substrates made of these materials are preferably coated on their surface with silane, poly-L-lysine, polystyrene or other suitable materials in order to facilitate the fixing of DNA.

DNA may be fixed in linear form onto the substrate by any method that allows individual DNA molecules to be fixed on a certain substrate as they are extended linearly. Exemplary methods include: the dynamic molecular combing (DMC) method in which DNA molecules are extended by slowly moving the liquid surface of a DNA solution over the substrate at constant speed (Michalet et al. (1997) Science 277:1518-1523); the water stream method in which DNA molecules are extended by applying a cover glass to a DNA solution spotted onto a substrate; the spin method in which DNA molecules are extended by spotting a DNA solution on a substrate as it rotates at high speed; the droplet sucking method in which DNA molecules are extended by spotting a DNA solution on a substrate and sucking up the droplets to create a water stream within the droplets; and the tilting method in which DNA molecules are extended by applying a DNA solution onto a substrate and tilting it to create a water stream (Fransz et al. (1996) Plant Journal 9:421-430). In the invention, the water stream method is preferably employed from the viewpoints of its operational simplicity and high efficiency.

In the water stream method, a DNA solution is spotted on a substrate and a cover glass is applied to generate a water stream such that individual DNA molecules are fixed as they are extended linearly. Specifically, a DNA sample such as an aqueous solution containing the DNA isolated and purified from cell nuclei is spotted on a slide glass and dried and, thereafter, a mounting fluid is spotted on a position slightly distant from the position where the DNA solution has been spotted and a cover glass is then applied to create a strong enough water stream to extend DNA molecules.

In order to ensure that the abasic sites on DNA are detected visually, a detecting means is employed that can identify the labelled abasic sites and which has a sufficiently high resolving power to detect the length of the DNA molecules of interest. Examples of the detecting means include an optical microscope, a CCD camera and a photomultiplier and these may be used either alone or in combination. In the invention, it is preferable to use a cooled CCD camera which, when combined with an optical microscope having a 100× objective lens, can provide a resolution of 100-50 nm.

The invention also provides an apparatus for implementing the above-described method for visual detection of abasic sites on DNA. This apparatus for visual detection of abasic sites on DNA is characterized by comprising (i) a fixing means by which a labelled DNA sample having labelled abasic sites on DNA is fixed in linear form onto a substrate and (ii) a detecting means for detecting the respective labelled abasic sites.

The following examples are provided for further illustrating the present invention but are by no means intended to limit its scope.

### EXAMPLES

### Example 1

### Detection of Fluorochrome

Cy5 bound streptavidin (Amersham Pharmacia Biotech, Buckinghamshire, England) was diluted 5,000-fold, spotted on a slide glass, dried, dispersed by the water stream method and fixed. The prepared sample was observed with a fluorescence microscope (Zeiss Axiovert, Carl Zeiss, Del.) equipped with a 100 W mercury lamp and a 100×, 1.4 NA Plan Apochromart objective lens (Zeiss Axiovert, Carl Zeiss, Del.), with images being captured by a thermoelectrically cooled (−20° C.) CCD camera with a pixel size of 67 nm×67 nm (Roper Scientific Inc., Trenton, N.J.) for fluorescence intensity measurement. The captured images were analyzed using the software MetaMorph (Version 4.5, Universal Imaging Corp., Downington, Pa.) and the emission of fluorescence was quantitated from the individual fluorescence probes.

The light emitted from a single fluorescence probe was focused on the CCD chip, where it formed a single point of light (ca. 7 pixel×7 pixel in size). To quantitate the fluorescence intensity per molecule of Cy5 bound streptavidin, at least 130 spots of light were formed, CCD pixel counts and the area of a point of light were integrated, and the background noise for the same area was subtracted from the integrated values.

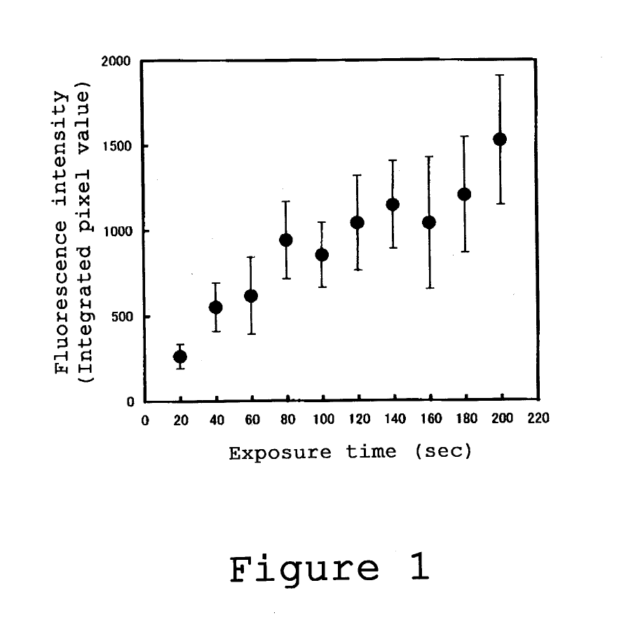

The results are shown in FIG. 1 (error bars representing standard deviation (s.d.), from which one can see that the fluorescence intensity increased with exposure time but some extinction was observed after the lapse of 100 seconds. It is therefore clear that with Cy5 bound streptavidin, luminescence is preferably observed within 80 seconds.

### Example 2

### Histogram of the Fluorescence Intensity from Fluorochrome Cy5 Bound dUTP and Streptavidin

Using Cy5-labelled dUTP (diluted 500,000-fold) and Sa-Cy5 (diluted 5,000-fold), the accuracy of quantification of fluorescence signals was tested by digital image analysis. The first specimen was dUTP having its molecule bound to one molecule of dye Cy5 and available as dUTP-Cy5 from Amersham Pharmacia Biotech. The second specimen Sa-Cy5 was streptavidin having its molecule bound to four molecules of dye Cy5 (fluorochrome-to-protein ratio=4:1).

By spotting and drying on slide glasses, dUTP-Cy5 and Sa-Cy5 were respectively adsorbed on the glass surface. The specimens were then mounted on the slides: dUTP-Cy5 and Sa-Cy5 were used as fluorescence standards for the quantification test and Sa-Cy5 was also used as a probe for detecting biotinylated DNA.

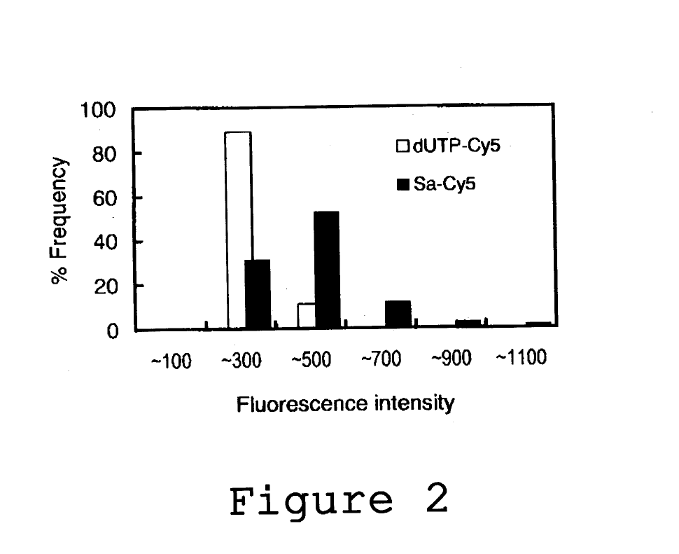

Fluorescence detection, image capture and analysis were performed using the method and apparatus described in Example 1. After photographing the images within a specified time (20 seconds), the fluorescence intensity of each point of light was calculated to make a comparison between dUTP and streptavidin (see FIG. 2).

The specimens showed average fluorescence intensities of 226.6±60.1 (dUTP-Cy5) and 387.1±151.6 (Sa-Cy5) and the resolving power was sufficiently high to identify one molecule of fluorochrome. The average fluorescence intensity from one molecule of Sa-Cy5 was about 1.7 times the value for dUTP-Cy5. By fluorescence intensity, one can tell whether the signal as detected is from a single molecules or from adjacent multiple molecules.

### Example 3

### Detecting Abasic Damage With Standard ARP-DNA Specimens Having Prescribed Numbers of Damaged Sites

In this example, ARP was used as a hydroxylamine derivative in order to check for the possibility of in situ visualization of abasic sites on DNA molecules. Standard ARP-DNA specimens having prescribed numbers (0, 20 and 40 sites) of damaged sites per 10⁵ bases (Dojindo, Kumamoto, Japan) were spotted undiluted on slide glasses, covered and incubated for 5 minutes. After removing the cover slips, the slide glasses were dried with air. Then, a drop (10 μL) of 1:10,000 diluted Cy5-bound streptavidin (Sa-Cy5, Amersham Pharmacia Biotech, Buckinghamshire, England) was deposited on the DNA spot, covered with a glass slip, reacted in a humidified chamber at 37° C. for 1 hour, washed three times with a Tris-sodium chloride buffer (TBST buffer; 50 mM Tris-HCl, pH 7.6, NaCl 300 mM, 0.05% (v/v) Triton X-100) and dried. The thus conditioned DNA samples were counter-stained with YOYO-1 (Molecular Probes Inc., Eugene, Oreg.) and observed with an optical microscope. Fluorescence detection, image capture and analysis were performed using the method and apparatus described in Example 1.

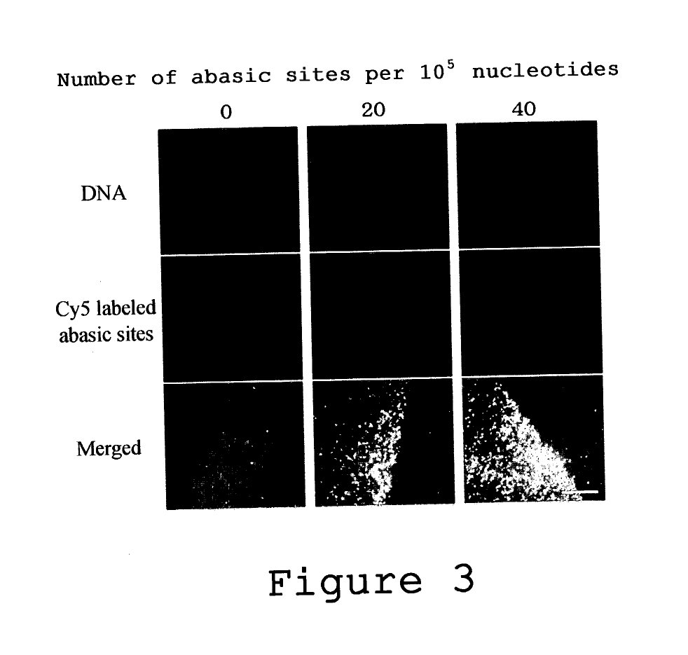

In spite of DNA fragmentation, fluorescence signals from Cy5 were detected at densities dependent on the number of damaged sites. See FIG. 3, in which the green signals indicate YOYO-1 stained DNA, the red signals indicate abasic sites labelled with Cy5-conjugated streptavidin, the yellow color shows localization of abasic sites on DNA, the column “0” refers to standard DNA (zero abasic sites/100,000 nucleotides) as a negative control, the column “20” refers to ARP-DNA standard DNA (20 abasic sites/100,000 nucleotides), the column “40” refers to ARP-DNA standard DNA (40 abasic sites/100,000 nucleotides), and the bar represents a length of 20 μm. In the standard ARP-DNA specimens, randomly fragmented DNA molecules were visible. Distinct fluorescence signals from Cy5 were observed in the standard DNA specimens having 20 and 40 abasic sites per 10⁵ nucleotides. The intensity of Cy5 signal was higher in the standard DNA specimen having 40 abasic sites per 10⁵ nucleotides than in the standard DNA specimen having 20 abasic sites per 10⁵ nucleotides. No significant signals were detected in the standard DNA specimen having zero abasic sites per 10⁵ nucleotides. Almost all Cy5 signals were localized in both standard DNA specimens having 20 and 40 abasic sites per 10⁵ nucleotides.

### Example 4

### Generation and Detection of Abasic Sites

In this example, in order to see whether abasic sites could be localized and subsequently counted on a single DNA molecule, abasic sites were generated on lambda-phage DNA and visually detected using ARP as a hydroxylamine derivative.

Lambda-DNA (Wako Pure Chemicals, Inc., Osaka, Japan) was dissolved in 10 mM sodium citrate buffer (pH 5.0) containing 100 mM sodium chloride and reacted at 70° C. for 0, 30, 60 or 90 minutes to generate target DNA specimens having abasic sites. Then, 10 μL of 10 mM biotinylated ARP (Dojindo, Kumamoto, Japan) in solution was mixed with 10 μL of each target DNA specimen in solution (50-100 ng/μL) and the mixture was incubated at 37° C. for 1 hour. The reaction solution was purified on a gel filtration spin column, further mixed with 10 μL of 1:5,000 diluted Cy5-bound streptavidin (Sa-Cy5) and incubated at 37° C. for 1 hour. The DNA-streptavidin conjugate was purified by loading the reaction solution on a gel filtration spin column, spotted on a substrate slide glass, dried, and processed by the water stream method to extend DNA molecules to make DNA samples. The thus prepared DNA samples were mounted together with 0.1 μM of YOYO-1. Fluorescence detection, image capture and analysis were performed using the method and apparatus described in Example 1.

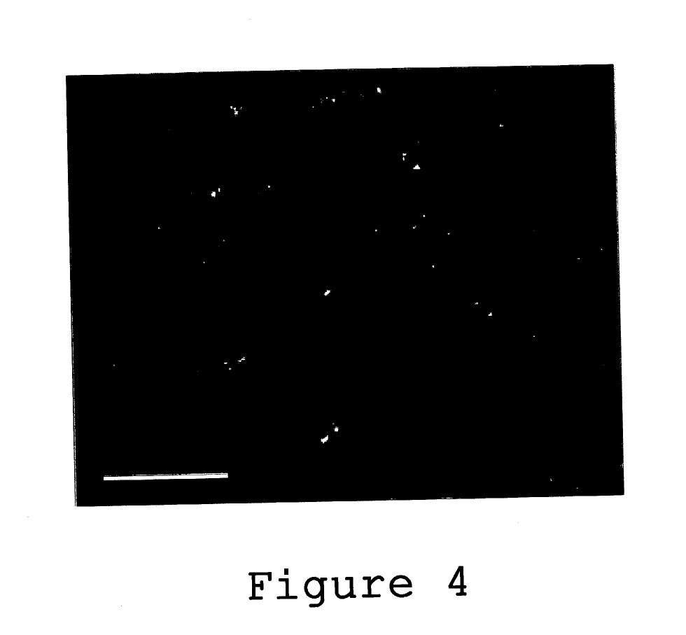

A representative example of the images taken with a CCD camera is shown in FIG. 4 (bar=20 μm). The lambda DNA molecules were individually extended in linear form and the Cy5 labelled abasic damaged sites were shown to be distributed among those DNA molecules.

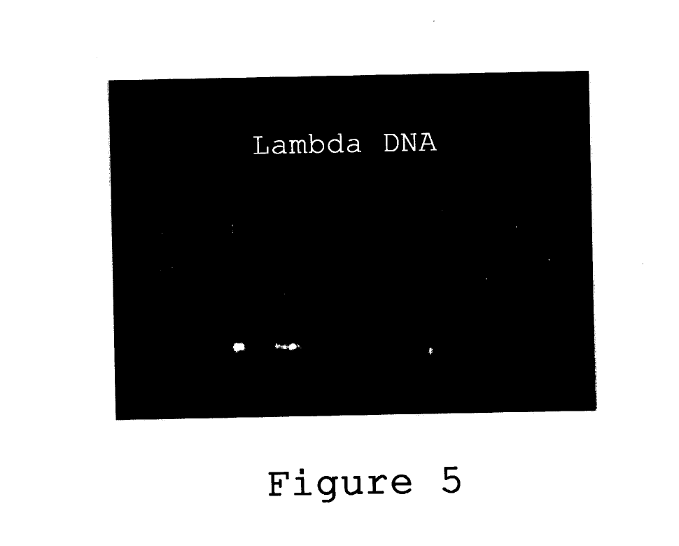

One of the DNA molecules is shown enlarged at a higher magnification in the lower part of FIG. 5. Obviously, four abasic sites are detected along the length of the molecule (in arbitrary orientation), showing that the number and location of abasic sites on a single DNA molecule could be clearly visualized (the upper part of FIG. 5). It was also clear that the method of the invention could analyze the distribution of specific sites at the submicron level.

### Example 5

### Sensitivity in the Detection of Abasic Damaged Sites

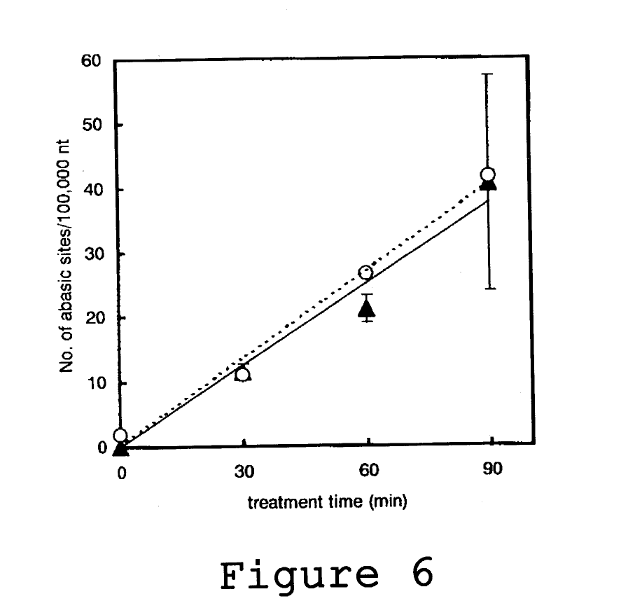

The method of visual detection employed in Example 4 was compared for sensitivity with the conventional enzyme assay using standard ARP-DNA specimens having prescribed numbers of damaged sites. The result is shown in FIG. 6, in which the error bars represent standard deviations (s.d.), the solid triangles and the solid line refer to the result of the visual detection, and the open circles and the dashed line refer to the result of the conventional assay. In the conventional assay, 41.7±0.65 abasic sites were induced per 100,000 nucleotides by a 90-min treatment with a citrate buffer. On the other hand, the method of visual detection according to the present invention could detect 40.6±16.8 abasic sites by a 90-min treatment. There was not found any occurrence of non-specific reaction between streptavidin and DNA.

The method of visual detection indicated by the solid line and the enzyme assay referred to by the dashed line showed a very good agreement in terms of detection sensitivity, thus confirming that the sensitivity of the invention method is almost comparable to that of the conventional method. This also shows the 1:1 correspondence between a visually detected abasic site and its entity.

### INDUSTRIAL APPLICABILITY

The present invention provides a novel method that is the first to achieve direct visualization and quantification of abasic sites on DNA by a single Sa-Cy5 molecule. This method is applicable to the DNA of all organisms including plants and animals and provides an accurate and simple identification of abasic sites.

An atomic force microscopy-based method for detecting abasic sites was recently developed (Sun, H. B. et al. (2001) Anal. Chem., 73, 2229-2232) but it could detect only two abasic sites on 250-bp DNA templates. The visualizing system of the invention, on the other hand, is based on optical microscopy and offers the advantage that it only requires the preparation of samples in a standard laboratory and allows them to be studied under liquid conditions.

In the method of the invention, abasic damage can be detected by a single Sa-Cy5 conjugate, so the sites of damage at neighboring bases can be counted in terms of fluorescence intensity. As is clear from FIG. 5, the method of the invention could resolve the distribution of specific sites at the submicron level. The detection of single molecular targets is currently limited by the sensitivity of probe molecules for the target and the efficiency of reaction between the probe and the target. According to the invention, DNA damage and modification can be detected by multi-color FISH.

Since abasic damage is a causative factor in carcinogenesis and aging of cells, the method of the invention can provide a sensitive assay technique as an aid in performing diagnoses of cancer tissues, checking individuals for their immunocompetence in DNA repair or assaying the safety of cancer control agents. The present invention can also be used as a convenient means for monitoring the effectiveness of a therapeutic treatment such as gene therapy on DNA having abasic sites. To be more specific, the therapeutic efficacy can be known by confirming that the abasic sites that were present before the treatment disappeared after the treatment.

If the method of the invention is performed using genetic markers known in the art, not only the number and location of abasic sites on specific genes or chromosomes but also their locations on the genome can be known simultaneously.

What is claimed is:

1. A method for visual detection of abasic sites on DNA, which comprises the steps of:
(i) labeling abasic sites on DNA; and
(ii) fixing a linear form of the labelled DNA onto a substrate; and
(iii) subsequently detecting the respective abasic sites on the fixed labelled DNA.

2. The method according to claim 1, wherein the labeling is performed with a fluorochrome.

3. The method according to claim 1 or 2, wherein the labeling of said abasic sites on DNA is performed by labeling their aldehyde group.

4. The method according to claim 1 or 2, wherein said aldehyde group is labeled using a hydroxylamine derivative which is reactive with an aldehyde group.

5. An apparatus for visual detection of abasic sites on DNA, which comprises:
(i) a fixing means by which a labelled DNA sample having labelled abasic sites on DNA is fixed in linear form onto a substrate; and
(ii) a detecting means for detecting the respective labelled abasic sites.
